(19) United States
(12) Patent Application Publication
(10) Pub. No.: US 2025/0177529 A1
(43) Pub. Date: Jun. 5, 2025

(54) ACOUSTIC ENRICHMENT OF ADOPTIVE CELL TRANSFERS

(71) Applicant: THE REGENTS OF THE UNIVERSITY OF COLORADO, A BODY CORPORATE, Denver, CO (US)

(72) Inventor: Charles W. Shields, Longmont, CO (US)

(21) Appl. No.: 18/842,951

(22) PCT Filed: Feb. 28, 2023
(86) PCT No.: PCT/US2023/014061

§ 371 Date: Aug. 30, 2024

Related U.S. Application Data
(60) Provisional application No. 63/315,729, filed on Mar. 2, 2022.

(51) Int. Cl. A61K 40/42; A61K 39/00; A61K 41/00; A61K 45/06; A61K 47/30; A61P 35/00

(52) U.S. Cl. CPC A61K 40/42; A61K 39/0011; A61K 41/00; A61K 45/06; A61K 47/30; A61P 35/00

(57) ABSTRACT

Patients with relapsing or refractory cancer have limited treatment options because the knowledge or presence of tumor-associated antigens is lacking. The proposed therapy mounts an antigen-independent anticancer response in a rapid, safe, and targeted manner due to its ability to stimulate innate immune cells in solid tumors, addressing a major, unmet major need in clinical oncology. A holistic system is disclosed capable of preparing and purifying cell samples for adoptive transfer into patients suffering from relapsing and refractory lymphomas and other solid cancers. An acoustofluidic device processes clinically relevant cell samples in a plug-and-play format, offering cell preparation speeds >100× faster than conventional CAR T-cell therapy.

### FIELD OF THE INVENTION

The present invention is related to the field of cancer immunotherapy. In particular, a composition and a method are disclosed to make and use a highly purified composition of a mononuclear cell: particle complex by acoustofluidic enrichment. The particles contain immunomodulatory drugs which reprogram tumor-associated immune cells to generate a robust immunological attack against solid tumors.

### BACKGROUND OF THE INVENTION

Chemotherapy remains the first line of treatment for most cancers due to cost, ease of administration, and flexibility to address most types of cancer. For patients who are relapsing or refractory to initial treatment, CAR T cell therapy is now available as a second line treatment for many eligible cancer patients.

However, CAR T cell therapies still suffer from several limitations. They: (i) are slow and expensive to manufacture (requiring 4-8 weeks to prepare, costing $500,000 per patient); (ii) rely on the knowledge and presence of tumor-associated antigens (in cases where tumor-associated antigens are unknown or poorly expressed, CAR T cell therapy is ineffective); and (iii) are ineffective against solid tumors.

What is needed in the art is a technology directly addressing these three challenges. This technology needs to be: (i) rapid and inexpensive (can be prepared in a few hours, potentially costing 100-1,000× less); (ii) agnostic to tumor-associated antigen expression; and (iii) effective against solid tumors.

### SUMMARY

The present invention is related to the field of cancer immunotherapy. In particular, a composition and a method are disclosed to make and use a highly purified composition of a mononuclear cell:particle complex by acoustofluidic enrichment. The particles contain immunomodulatory drugs which reprogram tumor-associated immune cells to generate a robust immunological attack against solid tumors.

In one embodiment, the present invention contemplates a composition comprising a substantially purified population of mononuclear cell:particle complexes that is substantially devoid of unbound particles. In one embodiment, the composition is a pharmaceutically acceptable composition. In one embodiment, the particles encapsulate an M1-macrophage polarizing drug. In one embodiment, the M1-macrophage polarizing drug is selected from the group consisting of resiquimod, motolimod, GS9620, CEP32496, BLZ945, OSI930, XL228, UNC2025, CELECOXIB, TMP195, TRICHOSTATIN A, IBET151, and INDOXIMOD. In one embodiment, the composition further comprises a checkpoint inhibitor. In one embodiment, the checkpoint inhibitor is anti-PD-1. In one embodiment, the particle further comprises an elastomeric polymer. In one embodiment, the elastomeric polymer is biodegradable. In one embodiment, the elastomeric polymer is a type of polyhydroxyalkanoate (PHA). In one embodiment, the particle has an anisotropic shape. In one embodiment, the particle is a negative acoustic contrast particle. In one embodiment, the mononuclear cell:particle complex has an acoustic contrast factor (φ) of greater than 0. In one embodiment, the unbound particles have an acoustic contrast factor (9) that is less than 0.

In one embodiment, the present invention contemplates a method, comprising: a) providing; i) a plurality of mononuclear cells; ii) a plurality of elastomeric particles; and iii) an M1-macrophage polarizing drug; b)combining said plurality of mononuclear cells, said plurality of elastomeric particles and said M1-macrophage polarizing drug to create a mixture comprising: i) a plurality of mononuclear cell:elastomeric particle complexes that encapsulate said M1-macrophage polarizing drug, and ii) a plurality of free, unbound, elastomeric particles that encapsulate said M1-macrophage polarizing drug; c) passing said mixture through an acoustic enrichment device to create a substantially purified population of mononuclear cell:elastomeric particle complexes that encapsulate said M1-macrophage polarizing drug that is substantially devoid of unbound elastomeric particles comprising said M1-macrophage polarizing drug; d) administering said substantially purified population of mononuclear cell:elastomeric particle complexes to a patient, such that a portion of said substantially purified population of mononuclear cell:elastomeric particle complexes embeds within a solid tumor, said solid tumor comprising tumor-associated immune cells; and e) releasing said M1-macrophage polarizing drug from said embedded substantially purified population of mononuclear cell:elastomeric particle complexes, thereby reprogramming said tumor-associated immune cells to reject said solid tumor in an antigen-agnostic manner. In one embodiment, the solid tumor is a cancer selected from the group consisting of bladder cancer, breast cancer, cervical cancer, colon cancer, endometrial cancer, esophageal cancer, gall bladder cancer, gastric cancer, glioblastoma, head and neck cancer, hepatocellular carcinoma, lymphoma, lung cancer, melanoma, mesothelioma, neuroendocrine cancer, ovarian cancer, pancreatic cancer, prostate cancer, renal cell carcinoma, and sarcoma. In one embodiment, the mononuclear cells are selected from the group consisting of mononuclear cells, macrophages, neutrophils, dendritic cells, NK cells, B cells, T cells, mast cells, mesenchymal stem cells, and their progenitor cells. In one embodiment, the M1-macrophage polarizing drug is selected from the group consisting of resiquimod, motolimod, GS9620, CEP32496, BLZ945, OSI930, XL228, UNC2025, CELECOXIB, TMP195, TRICHOSTATIN A, IBET151 and INDOXIMOD. In one embodiment, the administering further comprises a checkpoint inhibitor. In one embodiment, the checkpoint inhibitor is anti-PD-1. In one embodiment, the elastomeric particle comprises an elastomeric polymer. In one embodiment, the elastomeric polymer is biodegradable. In one embodiment, the elastomeric polymer is a type of polyhydroxyalkanoate (PHA). In one embodiment, the elastomeric particle has an anisotropic shape. In one embodiment, the elastomeric particle is a negative acoustic contrast particle. In one embodiment, the mononuclear cell:particle complex has an acoustic contrast factor (φ) of greater than 0. In one embodiment, the unbound particles have an acoustic contrast factor (φ) that is less than 0.

In one embodiment, the present invention contemplates a cancer vaccine composition comprising a plurality of negative acoustic contrast particles coated with a toll-like receptor (TLR) agonist and encapsulating an M1-macrophage polarizing compound. In one embodiment, the TLR agonist is bound to a tumor infiltrating dendritic cell. In one embodiment, the composition further comprises a medium capable of an intravenous (IV) injection or an intraperitoneal (IP) injection. In one embodiment, the cancer vaccine further comprises an anti-PD-1 checkpoint inhibitor.

In one embodiment, the present invention contemplates a method, comprising: a) providing: i) a patient in need of a cancer vaccine; ii) a cancer vaccine composition comprising a plurality of negative acoustic contrast particles coated with a toll-like receptor (TLR) agonist and encapsulating an M1-macrophage polarizing compound; b) administering said cancer vaccine composition to said patient thereby inducing an immunological response that generates anti-cancer antibodies; and c) preventing growth or metastasis of a cancer in said patient by said anti-cancer antibodies. In one embodiment, the administering is selected from the group consisting of an intravenous injection and an intraperitoneal injection. In one embodiment, the administering further improves tumor clearance. In one embodiment, the administering further induces protective immunity against molecularly similar tumors.

In one embodiment, the present invention contemplates a composition comprising 95% mononuclear cell:particle complexes and 5% particles. In one embodiment, the composition is a pharmaceutically acceptable composition. In one embodiment, the particles encapsulate a proinflammatory (M1)-macrophage polarizing drug. In one embodiment, the M1-macrophage polarizing drug is resiquimod. In one embodiment, the composition further comprises a checkpoint inhibitor. In one embodiment, the checkpoint inhibitor is anti-PD-1. In one embodiment, the particle comprises a soft (elastomeric) polymer. In one embodiment, the elastomeric polymer is a type of polyhydroxyalkanoate (PHA). In one embodiment, the particle is a negative acoustic contrast particle. In one embodiment, the mononuclear cell:negative acoustic contrast particle complex has an acoustic contrast factor (φ) that is greater than 0. In one embodiment, the elastomeric particle has an acoustic contrast factor (φ) that is less than 0.

In one embodiment, the present invention contemplates a method, comprising: a) providing; i) a plurality of mononuclear cells; ii) a plurality of elastomeric particles; and iii) an M1-macrophage polarizing drug; b) combining the plurality of mononuclear cells, plurality of elastomeric particles and the M1-macrophage polarizing drug to create a mixture comprising a plurality of mononuclear cell:particles, M1-macrophage polarizing drug encapsulated, particle complexes and free elastomeric particles, M1-macrophage polarizing drug encapsulated, particles; c) passing the mixture through an acoustic enrichment device to create a substantially purified mononuclear cell: elastomeric, M1-macrophage polarizing drug encapsulated, particle complex composition and an elastomeric, M1-macrophage polarizing drug encapsulated particle composition; d) administering a substantially purified mononuclear cell:particle, M1-macrophage polarizing drug encapsulated, particle complex composition to a patient, such that a portion of substantially purified mononuclear cell:particle, M1-macrophage polarizing drug encapsulated, particle complex composition embeds within a solid tumor comprising tumor-associated immune cells; and e) releasing the M1-macrophage polarizing drug from the embedded particle, thereby reprogramming tumor-associated immune cells to reject the solid tumors. In one embodiment, the solid tumor is a cancer including, but not limited to, breast cancer, melanoma cancer, sarcoma cancer or lymphoma cancer. In one embodiment, the mononuclear cells include, but are not limited to, monocytes, macrophages and/or mesenchymal stem cells. In one embodiment, the M1-polarizing drug is TMP195. In one embodiment, the composition further comprises a checkpoint inhibitor. In one embodiment, the checkpoint inhibitor is anti-PD-1. In one embodiment, the particle comprises an elastomeric polymer. In one embodiment, the elastomeric polymer is a type of PHA. In one embodiment, the particle is a negative acoustic contrast particle. In one embodiment, the mononuclear cell:negative acoustic contrast particle complex has an acoustic contrast factor (φ) greater than 0. In one embodiment, the elastomeric particle has an acoustic contrast factor (φ) that is less than 0.

In one embodiment, the present invention contemplates a cancer vaccine comprising a plurality of negative acoustic contrast particles coated with an agonist (e.g., a toll-like receptor (TLR)9 agonist such as CpG oligodeoxynucleotides (ODNs)) and an M1-polarizing compound. In one embodiment, the TLR9 agonists activate tumor-infiltrating dendritic cells (TIDCs). In one embodiment, the cancer vaccine is administered by intravenous (IV) injection. In one embodiment, the vaccine is co-administered with an anti-PD-1 checkpoint inhibitor. In one embodiment, the cancer vaccine improves tumor clearance. In one embodiment, the cancer vaccine induces protective immunity against molecularly similar tumors.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, fever, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or particle carrier) and a cell. Attachment may be reversible or irreversible. Such attachment includes, but is not limited to, covalent bonding, ionic bonding, Van der Waals attraction, and the like.

The term “drug” or “compound” as used herein, refers to any pharmacologically active substance capable of being administered which achieves a desired effect. Drugs or compounds can be synthetic or naturally occurring, small molecule, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), intravenous (IV) injection, intraperitoneal (IP) injection, intratumoral (IT) injection, subcutaneous (SC) injection, intramuscular (IM) injection, oral ingestion, transdermal patch, topical, inhalation, suppository etc. The administering can be performed using “a medium” including, but not limited to, a solution, a gel, a liquid or a semi-soft matrix that is capable of injection.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, means that an ingredient, substance or composition must be compatible chemically and/or toxicologically, with the other ingredients within a formulation, composition, and/or the animal being treated therewith. The term, “purified” or “isolated”, as used herein, may refer to a composition that has been subjected to separating forces (i.e., for example, fractionation) to remove various other components, and which composition substantially retains its expressed biological activity. Where the term “substantially purified” is used, this designation will refer to a composition in which the mononuclear cell:particle complex forms the major component of the composition, such as constituting about 50%, about 60%, about 70%, about 80%, about 90%, about 95% or more of the composition. A purified composition is not intended to mean that all trace impurities (e.g., unbound particles) have been removed.

The term “biocompatible”, as used herein, refers to any material does not elicit a substantial detrimental response in the host. In the context of this invention, biocompatibility is evaluated according to the application for which it was designed: for example; a bandage is regarded a biocompatible with the skin, whereas an implanted medical device is regarded as biocompatible with the internal tissues of the body. Preferably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “biostable” as used herein, refers to any material that remains within a physiological environment for an intended duration resulting in a medically beneficial effect.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “monodisperse particles”, as used herein include a population of particles wherein at least about 60% of the particles in the population, more preferably 75% to 90% of the particles in the population, or any integer in between this range, fall within a specified particle size range. A population of monodispersed particles deviate less than 10% rms (root-mean-square) in diameter and preferably less than 5% rms.

The term “mononuclear cell” as used herein refer to blood cells that have a single, round nucleus, such as lymphocytes and monocytes. When isolated from circulating blood, they are called peripheral blood mononuclear cells (PBMC), but other sources exist, such as the umbilical cord, spleen, and bone marrow. For example, mononuclear cells include, but are not limited to, lymphocytes, monocytes, natural killer cells (NK cells) and/or dendritic cells.

The term “macrophage polarizing drug” as used herein, refers to any compound that induces macrophages to produce distinct functional phenotypes. For example, macrophages can be polarized into classically activated (M1) and alternatively activated (M2) macrophages. For example, M1-macrophage polarizing drugs include, but are not limited to, resiquimod, motolimod, GS9620, CEP32496, BLZ945, OSI930, XL228, UNC2025, CELECOXIB, TMP195, TRICHOSTATIN A, IBET151 and INDOXIMOD.

The term “tumor-associated macrophages (TAMs)” refers to a class a macrophages that are typical for their protumoural functions like promotion of cancer cell motility, metastasis formation and angiogenesis. Their formation is dependent on microenvironmetal factors which are present in developing tumour. TAMs produce immunosuppressive cytokines like IL-10, TGFβ and PGE2. Targeting of TAMs is contemplates herein as a therapeutic strategy against cancer that may involve inducing TAMs from an M2 to an M1 phenotype.

The term “substantially devoid” as used herein refers to a negligible concentration of a component in a composition such that the component contributes little or nothing to the properties of the composition. For example, a negligible concentration includes, but is not limited to less than 5%, less than 4%, less than 3%, less than 2%, less than 1%, less than 0.5% less than 0.25%, less than 0.1% and or approximately or actually 0%.

### BRIEF DESCRIPTION OF THE FIGURES

FIG. 1 illustrates an overview of one embodiment of an acoustofluidic enrichment method.

FIG. 1A: Negative acoustic contrast particles (NACPs) loaded with TMP195 and coated with CpG ODNs bind to mononuclear cell.

FIG. 1B: Acoustic purification of mononuclear cell:NACP complexes using a high throughput device.

FIG. 1C: Co-administration of acoustically purified mononuclear cell:NACP complexes and anti-PD-1 in mice with established CD19-sparse lymphoma.

FIG. 2 presents one embodiment of a multi-parallel acoustofluidic enrichment instrument to purify mononuclear cell-NACP populations for adoptive cellular transfers using a single microfluidic device with parallel channels for high throughput processing.

FIG. 3 presents exemplary data showing the production of discoidal NACPs.

FIGS. 3A-C: An illustration of microcontact printing production of discoidal particles. FIG. 3D: A representative photomicrograph of an array of microprinted discoidal NACPs.

FIGS. 3E-F: Confocal (left) and scanning electron microscope (right) images of monocytes attached to discoidal NACPs.

FIG. 4 presents exemplary data showing the acoustofluidic focusing of cells and NACPs.

FIG. 4A: Acoustic separation of unbound NACPs from cell-bound NACPs.

FIG. 4B: Images of cells in a device at 100 μL/min with the standing wave off (top) and on (bottom).

FIG. 4C: Images of PHA-based NACPs containing a model drug in a device at 100 μL/min with the standing wave off (top) and on (bottom).

FIG. 5 presents exemplary data showing in vivo particle targeting to lipopolysaccharide (LPS)-inflamed mouse skin. 5 μg LPS in 30 μL saline was injected intradermally into the lower left mammary pad of BALB/c mice. Particles and cells were injected systemically through the lateral tail vein. Biodistribution data was collected 6 hrs later by harvesting organs and fluorescence intensity.

FIG. 5A: Free particles mostly accumulated in the liver and spleen.

FIG. 5B: Monocyte-bound particles showed a ˜9-fold increase in targeting efficiency to inflamed skin and a 3-fold decrease in accumulation in the liver and spleen.

FIG. 6 presents exemplary data showing the percentage of surface-associated microdiscs that evaded phagocytosis over 5 days as compared to spheres of similar volume (n=5 repeat studies).

FIG. 7 presents exemplary data showing primary macrophage polarization in various conditions.

FIG. 7A: Normoxia (i.e., standard cell culture conditions) without interferon gamma (IFNγ).

FIG. 7B: Normoxia with IFNγ.

FIG. 7C: Tumor-promoting conditions (i.e., in hypoxia (1% 02) and 10 vol. % tumor (4T1)-conditioned media) and bound to IFNγ-containing particles after 24 hrs.

FIG. 8 present exemplary data showing PHA-based NACPs (red) bound to RAW 264.7 cells (blue, DAPI) separating from unbound NACPs (red) in an acoustofluidic instrument.

### DETAILED DESCRIPTION OF THE INVENTION

The present invention is related to the field of cancer immunotherapy. In particular, a composition and a method are disclosed to make and use a highly purified composition of a mononuclear cell:particle complex by acoustofluidic enrichment. The particles contain immunomodulatory drugs which reprogram tumor-associated immune cells to generate a robust immunological attack against solid tumors.

In one embodiment, the present invention contemplates a system and a method that induces TAM repolarization and cytotoxic T lymphocyte (CTL) induction. In one embodiment, mononuclear cell:NACP complexes slowly release an M1-macrophage polarizing drug (e.g., TMP195) deep within solid tumors to repolarize TAMs toward M1-macrophage associated phenotypes, thereby rejecting tumor growth and development. In one embodiment, mononuclear cell:NACP complexes stimulate TLR9 receptors on TIDCs to activate their presentation of antigens and migration to naïve T-cells drainage lymph nodes to induce CTL. In one embodiment, the method further comprises an antigen-specific attack against the solid tumors with the primed CTLs and administering a checkpoint inhibitor (e.g., anti-PD-1).

### I. Adoptive Cellular Therapies

Adoptive cellular therapies have shown outstanding progress in cancer therapy. Grupp et al. “Chimeric antigen receptor-modified T cells for acute lymphoid leukemia” N Engl J Med. 2013; 368(16):1509-18; and Maude et al., “Chimeric antigen receptor T cells for sustained remissions in leukemia” N Engl J Med. 2014; 371(16):1507-17. Unlike chemotherapeutic drugs, which affect tissues indiscriminately, immune cells have the capacity to treat diseased areas in a highly specific manner.

### A. Chimeric Antigen Receptor (CAR) T-Cells

The state of the art for adoptive cellular therapies is presently believed to be CAR T-cell therapy. CAR T-cell therapy has successfully treated patients with cancers (e.g., non-Hodgkin lymphoma (NHL)) that are relapsing or refractory to first-line treatment. Schuster et al. “Chimeric Antigen Receptor T Cells in Refractory B-Cell Lymphomas” N Engl J Med. 2017; 377(26):2545-54. Neelapu, et al. “Axicabtagene Ciloleucel CAR T-Cell Therapy in Refractory Large B-Cell Lymphoma” N Engl J Med. 2017; 377(26):2531-44. Kochenderfer, et al. “Long-Duration Complete Remissions of Diffuse Large B Cell Lymphoma after Anti-CD19 Chimeric Antigen Receptor T Cell Therapy” Mol Ther. 2017; 25(10):2245-53. However, CAR T-cell therapies have several limitations, they: (i) are slow and expensive to manufacture; (ii) are inhibited by suppressive cell populations such as tumor-associated macrophages (TAMs) with M2-associated phenotypes; and (iii) rely on the high expression of tumor-associated antigens. Thus, there is a broad need to develop therapies that are rapid, scalable, and agnostic to the expression of tumor-associated antigens in solid tumors.

### B. Negative Acoustic Contrast Particles

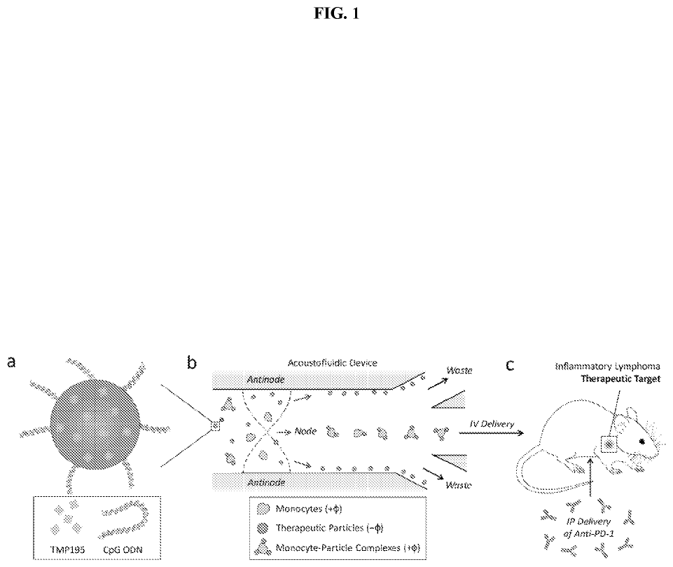

In one embodiment, the present invention contemplates an integrated system to rapidly prepare a mononuclear adoptive cell-immunomodulatory particle complex. See, FIG. 1A. It is believed that these particles potentiate a robust anticancer response against NHLs. For example, it has been reported that particles bound to, or internalized within, mononuclear cells facilitate transport deep into solid tumors. Singh et al. “Harnessing cells to deliver nanoparticle drugs to treat cancer” Biotechnol. Adv. (2019).

In one embodiment, the mononuclear adoptive cell-immunomodulatory particles are negative acoustic contrast particles (NACPs) and comprise elastomeric, biodegradable polymers. In one embodiment, the NACPs permit a gentle acoustic separation (e.g., acoustic enrichment) of unbound (e.g., free) immunomodulatory particles from particles bound to mononuclear cells for adoptive transfer into cancer patients. See, FIG. 1B; and Shields I V et al., “Elastomeric negative acoustic contrast particles for capture, acoustophoretic transport, and confinement of cells in microfluidic systems” Langmuir. 2014; 30(14):3923-7. Preventing such unbound NACPs from being administered to a patient minimizes off-target accumulation and toxicity. See, FIG. 1C. Although it is not necessary to understand the mechanism of an invention, it is believed that NACP elasticity allows them to evade phagocytosis. Anselmo et al., “Elasticity of Nanoparticles Influences Their Blood Circulation, Phagocytosis, Endocytosis, and Targeting” ACS Nano. 2015; 9(3):3169-77.

Consequently, the NACPs that are bound to mononuclear cells infiltrate solid tumors more efficiently than unbound particles, whereupon they permit a slow release of drugs in the tumor microenvironment to: (i) repolarize tumor-associated macrophages (TAMs); and (ii) activate dendritic cells (DCs) to educate antigen-specific cytotoxic T lymphocytes (CTLs).

### Elastomeric Particles

The use of elastomeric, biodegradable NACPs provides a superior treatment benefit over conventional CAR T-cell systems by: i) enabling rapid acoustic preparation; ii) trafficking deep into tumors to reject CD19-sparse lymphomas; and iii) releasing drugs for sustained periods to reprogram tumor-associated immune cells. The data presented herein demonstrates that a particle:drug:mononuclear cell complex have the potential to infiltrate tumors five-to-ten-fold more efficiently than particles alone. Thus, particle:drug:mononuclear cell complexes improve drug biodistribution and reduces toxicity.

Elastomeric particles have been reported to provide biomimetic properties. Wu et al., “Biomedical Application of Soft Nano-/Microparticles” In: Dobashi R K, editor. Nano/Micro Science and Technology in Biorheology. London: Springer; 2015. p. 261-94. For example, elastomeric particles have been shown to resist cellular phagocytosis. Garapaty et al., “Tunable particles alter macrophage uptake based on combinatorial effects of physical properties” Bioeng Transl Med. 2017; 2(1):92-101. This resistance to phagocytosis allows the particles to remain associated with the surfaces of the cells to which they are attached to release drugs into the extracellular space within solid tumors and more effectively reprogram tumor-associated immune cells.

Furthermore, it is known that elastomeric particles migrate to the pressure antinodes of an acoustic standing wave. Johnson et al., “Elastomeric microparticles for acoustic mediated bioseparations” Journal of Nanobiotechnology. 2013; 11(22):1-19. Consequently, the disclosed NACPs comprise elastomeric particles that resist phagocytosis and undergo negative acoustophoresis.

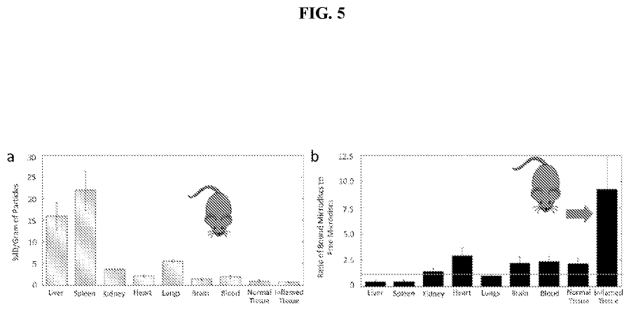

A rapid and efficient separation of unbound NACPs from NACPs bound to mononuclear cells (e.g., monocytes) creates a highly purified mononuclear cell-NACP composition. The data presented herein demonstrates that this highly purified mononuclear cell-NACP composition has superior clinical efficacy. For example, a landmark survey analyzing drug delivery platforms over 10 years found that only 0.7% of injected nanoparticles infiltrate solid tumors when injected systemically (e.g., IV). Stefan-Wilhelm et al. “Analysis of nanoparticle delivery to tumours” Nature Reviews Materials. 2016; 1(15):1-12. Furthermore, most nanoparticles accumulate in the liver, spleen. and lungs, generating toxicity. Although it is not necessary to understand the mechanism of an invention, it is believed that mononuclear cell-mediated transport of particles allows for more efficient accumulation of particles into tumors due the abundant expression of chemokine receptors on mononuclear cells. Kohli et al., “Key chemokines direct migration of immune cells in solid tumors” Cancer Gene Ther. (2021). For example, it was found that ˜7.9% of injected monocytes infiltrate 4T1 (breast) tumors in mice. Combes et al., “Off-Target and Tumor-Specific Accumulation of Monocytes, Macrophages and Myeloid-Derived Suppressor Cells after Systemic Injection” Neoplasia. 2018; 20(8):848-56. It was suggested that this mononuclear cell accumulation could be further improved by adjusting sample concentrations and injection timings, this tumor-targeting performance is ˜10-times more efficient (i.e., targeted) than free particles. See, FIG. 5B.

It has been shown that elastomeric particles comprise useful acoustic properties, evade phagocytosis, encapsulate immunomodulatory payloads and bind to cells. Also reported is a method to synthesize NACPs in bulk quantities using a unique nucleation and growth technology. The resultant NACPs can be monodisperse, have modifiable surface chemistries, and tunable acoustic contrast factors (φ). Shields I V et al., “Nucleation and growth synthesis of siloxane gels to form functional, monodisperse, and acoustically programmable particles” Angew Chem Int Ed Engl. 2014; 53(31):8070-3; Li et al., “Rapid capture of biomolecules from blood via stimuli-responsive elastomeric particles for acoustofluidic separation” Analyst. 2021; 145(24):8087-96; U.S. Pat. Nos. 9,797,897, 10,238,586, and U.S. Patent App. 62/616,519 (all herein incorporated by reference).

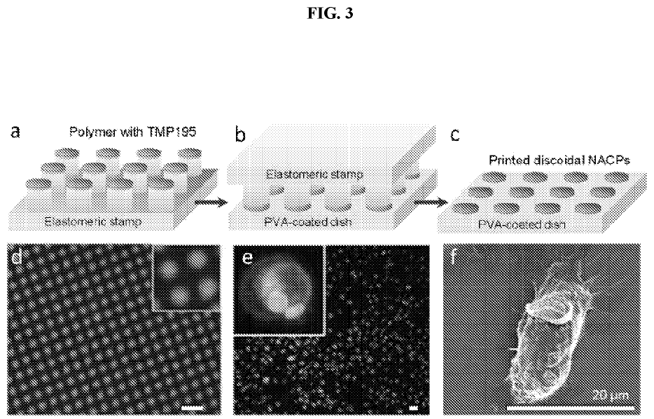

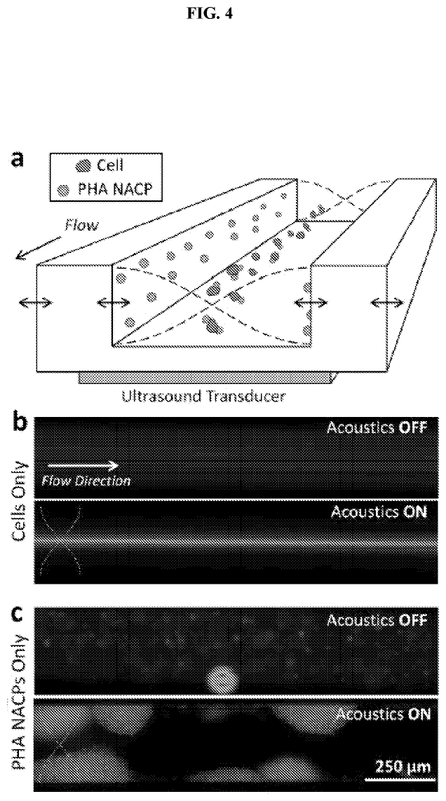

The data presented herein demonstrates preparation of spherical and discoidal particles made from polyhydroxyalkanoate (PHA), which is a biodegradable elastomer. See, FIG. 3A-D for discoidal particles and FIG. 4B-C for spherical particles. PHA has properties including, but not limited to, a bulk modulus of less than 1.00 GPa and an acoustic contrast factor (φ) of less than 0. These properties result in an attraction of PHA particles to pressure antinodes when not bound to cells. See, FIG. 4.

An alternative to PHA is a biodegradable elastomer that is highly elastomeric (i.e., 0.5-1.0 MPa), such as poly(glycerol sebacate) (PGS). While this polymer is less studied than PHA, it may improve phagocytosis resistance. Louage et al., “Poly(glycerol sebacate) nanoparticles for encapsulation of hydrophobic anti-cancer drugs” Polymer Chemistry. 2017; 8(34):5033-8.

### Phagocyte Evasion

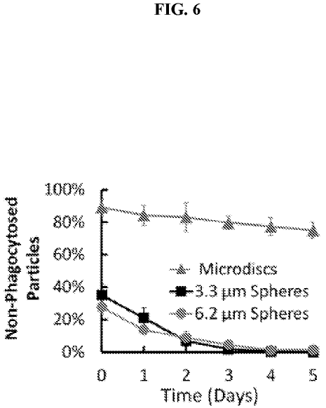

It has been shown that elastomeric particles of anisotropic shape strongly resist phagocytosis due to the frustration of actin filament formation. Champion et al., “Role of target geometry in phagocytosis” Proc Natl Acad Sci USA. 2006; 103(13):4930-4. Furthermore, the present data confirms earlier reports that microdiscs made from biodegradable polymers can readily attach to myeloid cells (e.g., macrophages) and resist phagocytosis for at least 5 days. See, FIG. 6; and Shields I V et al., “Cellular backpacks for macrophage immunotherapy” Science Advances 2020; 6(18):eaaz6579. The microdiscs can be made using a microcontact printing technique that is scalable, biocompatible, and reproducible. In one embodiment, a PHA particles comprises a cell-adhesive layer, wherein said cell-adhesive layer comprising a layer-by-layer (LBL) assembly of hyaluronic acid (HA) and poly(allylamine) hydrochloride (PAH). In one embodiment, the cell-adhesive layer binds approximately 86.9% of the cells, as compared to 63.4% without the cell-adhesive layer.

Phagocytic suppression allows NACPs to travel deep into the tumor microenvironment to impart therapeutic effects. It is increasingly recognized that particle ingestion by phagocytes (e.g., macrophages, monocytes) can be suppressed by the rational design of shape or elasticity. An elastomeric particle may include, not limited to, non-spherical particles (e.g., triangular, square, and hexagonal microdiscs) using monolithic fabrication and/or microcontact printing. See, FIG. 3. An elastomeric particle may include, but not limited to, a spherical elastomeric particle comprising a silicone with φ≈−1.5 and PHA with φ≈−7.3. See, FIG. 4. Shields I V, et al. “Nucleation and growth synthesis of siloxane gels to form functional, monodisperse, and acoustically programmable particles” Angew Chem Int Ed Engl. 2014; 53(31):8070-3.

Discoidal particles have been reported that are 7.5 μm across and 1.5 μm thick, attach to primary bone marrow-derived macrophages and evade phagocytosis for at least 5 days. 77.3% of the disc-shaped particles (Volume=49.8 μm3) remained surface-bound after 5 days, compared to <5% of spheres of a similar size (Volume=124.8 μm³). See, FIG. 6; and Shields IV et al., “Cellular backpacks for macrophage immunotherapy” Science Advances 2020; 6(18):eaaz6579. The data presented herein, found that PHA microdiscs without a bound drug attaches to mononuclear cells after incubation of only 1.5 hrs. See, FIG. 3E-F. These data shows that PHA elastomeric particles resist phagocytosis.

### Phenotype Programming

As discussed above, discoidal elastomeric particles can encapsulate inflammatory cytokines (i.e., IFNγ) and polarize the cells to which they are bound. Such particles induced a shift in polarization toward M1-like phenotypes, indicated by increased expressions of inducible nitric oxide synthase (iNOS), major histocompatibility complex (MHC) II, and CD80/86 compared to controls. Most of all, iNOS expression was 629.3-fold higher in cells carrying microdiscs encapsulating IFNγ, compared to only 2.4-fold and 1.3-fold higher in cells with blank microdiscs and an equivalent dose of IFNγ, respectively. Macrophages maintained their polarization for 5 days, even when challenged by hypoxia (1% O₂) and tumor-conditioned media (i.e., 10 vol. % tumor (4T1)-conditioned media).

After administering cells with IFNγ microdiscs into 4T1 breast tumor-bearing mice, significant increases in iNOS and MHCII expression were observed in TAMs compared to controls. These mice showed reduced tumor growths and metastatic burdens compared to controls, supporting a mechanism of tumor-associated macrophage (TAM) repolarization. The TAM-repolarizing drug (TMP195) is a small molecule relative to IFNγ, consequently it is easier to stabilize and deliver, thereby resulting in more potent immune responses.

### Mononuclear Cell Trafficking to Solid Tumors

It has been shown that mononuclear cells can transport discoidal particles to sites of inflammation (i.e., lipopolysaccharide (LPS)-inflamed skin). Compared to free particles, which accumulated primarily in the liver and spleen, mononuclear cell-bound particles exhibited a ˜9-fold increase in accumulation in LPS-inflamed skin. In contrast, only a modest increase in accumulation occurred in non-LPS-inflamed skin. In parallel, the relative change in particle accumulation in the liver and spleen decreased by ˜3-fold due to mononuclear cell transport consistent with particle localization in tumors. See, FIG. 5A-B. Other reports have shown that similar disc-shaped microparticles bound to mononuclear cells can target inflammation in the brain. Klyachko et al., “Macrophages with cellular backpacks for targeted drug delivery to the brain” Biomaterials. 2017; 140:79-87.

### Particle Properties

Tumors generate sizable degrees of inflammation, thereby recruiting large numbers of myeloid cells like macrophages and monocytes through cellular chemotaxis. Once incorporated into a tumor, mononuclear cells encounter many tumor cells, stromal cells, and tumor-associated macrophages (TAMs). The role of tumor-associated immune cells in tumor growth or rejection is dependent upon their phenotypic polarization. Murdoch et al., “Mechanisms regulating the recruitment of macrophages into hypoxic areas of tumors and other ischemic tissues” Blood. 2004; 104(8):2224-34; Mosser et al., “Exploring the full spectrum of macrophage activation” Nat Rev Immunol. 2008; 8(12):958-69. By labeling mononuclear cells with particles that slowly release M1-macrophage polarizing drugs (e.g., TMP195) mononuclear cells can traffic particles deep within lymphatic tumors to guide the immune rejection of those tumors. The data presented herein demonstrates cellular transport of the presently disclosed particles. See, FIG. 5.

Particle shape and elasticity are exemplary parameters that determine particle resistance to phagocytosis and compatibility with acoustic purification. Preferred physical properties of biodegradable NACPs include, but are not limited to, residence time on cellular surfaces and/or transport to lymphatic tumors. Although it is not necessary to understand the mechanism of an invention, it is believed that: (i) spherical and discoidal NACPs attach to mononuclear cells and evade phagocytosis for at least 48 hrs and (ii) injected NACP-mononuclear cell complexes infiltrate and attack tumors through repolarization of TAMs.

The shape of NACPs may be controlled by the use of different methods, for example, homogenization or microcontact printing. Homogenization results in the production of spherical particles. Briefly, low concentrations of PHA are dissolved in an organic solvent (e.g., dichloromethane (DCM)) with an M1-macrophage polarizing drug; preferably the solution is added dropwise to an aqueous solution with a surfactant, then the solution is homogenized with a sonicator for 5 minutes. Stirring the solution overnight removes the DCM. See, FIG. 4C. Microcontact printing results in the production of microdiscs. Briefly, photolithography defines an array of holes in a photoresist material on silicon wafers. For example, an inverse mold (i.e., “elastomeric stamp”) can be created from polydimethylsiloxane. See, FIG. 3A. Subsequently, a mixture of PHA, DCM and an M1-polarizing drug is spin coated onto the stamp. The coated stamp is then pressed onto a substrate coated with a layer of polyvinyl alcohol (PVA). See, FIG. 3D. The resultant printed particles can be detached by the addition of water. The data presented here using fluorescence microscopy show that the discoidal PHA particle microdiscs made from poly(lactic-coglycolic acid; PLGA) resist phagocytosis for 5 days. See, FIG. 6.

Spherical NACPs made by homogenization produces a polydisperse size distribution. Thus, a selection of a narrow size distribution (i.e., coefficient of variance <20%) can be achieved by differential centrifugation or serial filtration (e.g., 1-5 m in diameter). In contrast, microdise-shaped NACPs made by microcontact printing produces a monodisperse size distribution. The use of photomasks results in the ability to make discoidal particles of many different sizes. The thickness of discoidal particles may also be controlled by modulation of the spin coating parameters. In one embodiment, the present invention contemplates discoidal NACPs of 3-8 μm across and 0.2-2 μm thick.

The surfaces of the NACPs can be coated with other drugs (e.g., biological drugs such as proteins, antibodies, nucleic acids), including CpG oligodeoxynucleotides (ODNs). NACPs are functionalized with materials that facilitate incorporation of CpG ODNs on the NACP surface. For example, NACPs are coated with a CpG-based LBL. CpG is negatively charged, whereas poly(allylamine) hydrochloride (PAH) is positively charged, enabling their electrostatic self-assembly on particle surfaces. The data shown herein demonstrates that LBL-coated microdiscs bind to monocytes. See, FIG. 5. Alternatively, NACPs are coated with a metal-phenolic network (MPN) formed from the polymerization of tannic acid (TA), thereby making a “sticky” polyphenol. Guo et al., “Modular assembly of superstructures from polyphenol-functionalized building blocks” Nat Nanotechnol. 2016; 11(12):1105-11. It has recently been shown that particles can be coated with an MPN network, which increases their binding to monocytes 3-fold compared to controls. Zhao et al., “Engineering of Living Cells with Polyphenol-Functionalized Biologically Active Nanocomplexes” Adv Mater. 2020:e2003492. In one embodiment, the present invention contemplates NACPs comprising CpG ODNs with or without a polymerized MPN layer. Lin et al., “Gold nanoparticle delivery of modified CpG stimulates macrophages and inhibits tumor growth for enhanced immunotherapy” PLoS One. 2013; 8(5):e63550.

In one embodiment, the present invention contemplates a plurality of NACPs, wherein each NACP comprises a different shape, size and/or surface treatment. Mononuclear cell binding of each of these NACPs can be quantitated by flow cytometry subsequent to fluorescent dye incorporation. See, FIG. 4C. Phagocytosis evasion properties can be evaluated using confocal microscopy and pH-sensitive dyes (e.g., pHrodo). Miksa et al., “A novel method to determine the engulfment of apoptotic cells by macrophages using pHrodo succinimidyl ester” J Immunol Methods. 2009; 342(1-2):71-7.

NACPs are loaded with M1-macrophage polarizing drugs, such as TMP195, to potentiate a robust shift in macrophage polarizations. Different concentrations of these agents may be used, for example, between 0.1-15 wt. %, at 50 mg/kg (˜10⁶ NACPs injected/mouse). Like TMP195, most M1-polarizing drugs are hydrophobic, which renders them easily encapsulated into hydrophobic polymers (e.g., PHA). Elastomeric particles may also encapsulate drugs including, but not limited to, small hydrophobic molecules, for example, resiquimod, retinol, ingenol mebutate, and betamethasone as well as hydrophilic proteins like IFNγ that induce proinflammatory responses. Encapsulation of many hydrophobic drugs into elastomeric particles up to 10 wt. % by solids has been demonstrated. Shields I V et al., “Encapsulation and controlled release of retinol from silicone particles for topical delivery” J Control Release. 2018; 278:37-48.

NACP loading efficiency and release kinetics of M1-polarizing drugs can be measured using tests known in the art. For example, NACPs are dissolved in an organic solvent (e.g., DCM) and the total amount of drug encapsulated will be compared with the total amount of drug added to the reaction to determine the loading efficiency. Release kinetics are measured by incubating the particles in an end-over-end rotator at 37° C. for 5 days. At each time point, samples are centrifuged, media collected and stored at −80° C. Standard curves (following Beer-Lamberts law) are used to determine the amount of M1-macrophage polarizing drug released between each time point, whereupon the cumulative release profile can be determined using UV-vis spectroscopy or liquid chromatography-mass spectrometry (LC-MS).

In particular, the potency of TMP195-containing NACPs can be tested by adding different concentrations of NACPs to in vitro cultures of primary bone marrow-derived macrophages (BMDMs) isolated and differentiated from mice (e.g., BALB/c). NACP concentrations are chosen that correspond directly with BMDMs in tumor-mimicking conditions (e.g., 1% O₂ and in tumor-conditioned media from cultures of CD19-depleted A20 lymphoma tumor cells). Alternatively, encapsulation of resiquimod (R-848), a potent toll-like receptor (TLR)7/8-agonist with a maximum tolerated dose (MTD) of 5 mg/kg, produces an effect that is similar to TMP195. Rodell et al., “TLR7/8-agonist-loaded nanoparticles promote the polarization of tumour-associated macrophages to enhance cancer immunotherapy” Nature Biomedical Engineering. 2018; 2:578-88; Vinod et al., “High-capacity poly(2-oxazoline) formulation of TLR 7/8 agonist extends survival in a chemoinsensitive, metastatic model of lung adenocarcinoma” Science Advances. 2020; 6(25):eaba5542. 7; and Ho et al., “Adjuvants Enhancing Cross-Presentation by Dendritic Cells: The Key to More Effective Vaccines?” Front Immunol. 2018; 9:2874.

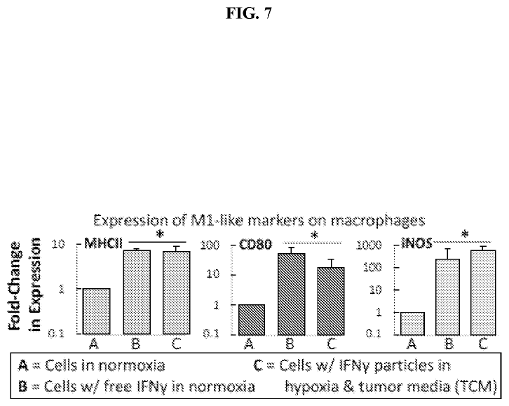

BMDMs are then isolated, differentiated, and cultured using known methods. After 24, 72, and 96 hrs, media is collected and screened for excreted cytokines (e.g., TNFα, IL-1β, IL-6, IL-12, and IL-18) using ELISA as an indicator a phenotypic shift toward M1-associated macrophage polarizations. Cells are also harvested and screened for surface and intracellular activation markers of M1-associated macrophage polarizations (e.g., CD80/86 and iNOS). The data presented herein show that similar particles containing IFNγ can stimulate cultures of BMDMs. See, FIG. 7. These data suggest that PHA-based NACPs containing TMP195 potently polarize macrophages.

### II. Acoustic Particle Enrichment

Although it is not necessary to understand the mechanism of an invention, it is believed that acoustic enrichment ensures that only material particle:mononuclear cell complexes are injected into patients, ensuring optimal biodistribution. This technology enables the rapid and cost-effective treatment of solid tumors in a way that addresses the main pitfalls of CAR T-cell therapy, which is the current gold standard for treating relapsing and refractory cancers.

In one embodiment, the present invention contemplates an acoustic device configured to rapidly separate unbound (e.g., free) particles from particles bound to a biological cell (e.g., a mononuclear cell) to provide a highly purified composition of particle:cell complexes. In one embodiment, the acoustic device does not include any magnetic or paramagnetic materials. The presently disclosed acoustic device is a modification based upon previously reported principles. Kaduchak et al., “Application of acoustic radiation pressure to align cells in a commercial flow cytometer” Proceedings of Meetings on Acoustics. 2013:1-5.

Briefly, suspended particles and cells either focus along the pressure node or antinodes of an acoustic standing wave. See, FIG. 1B. The focus position is predicted by the acoustic contrast factor determinant, 9, which is a dimensionless quantity given by:

$${φ}_{p} = \frac{5 {ρ}_{p} - 2 {ρ}_{f}}{2 {ρ}_{p} + {ρ}_{f}} - \frac{{β}_{p}}{{β}_{f}} ,$$

in which the variables ρ and β represent density and compressibility and the subscripts p and f represent the suspended object (e.g., particle or cell) and the fluid, respectively. If φ>0, as is the case with virtually every mammalian cell, objects migrate to the pressure node (center of the acoustic standing wave). If φ<0, as is the case with soft (elastomeric) particles, objects migrate to the pressure antinodes, hence their classification as negative acoustic contrast particles (NACPs). This phenomenon is well-known and has been demonstrated by multiple groups. Petersson et al., “Separation of lipids from blood utilizing ultrasonic standing waves in microfluidic channels” Analyst. 2004; 129(10):938-43; Nilsson et al., “Acoustic control of suspended particles in micro fluidic chips” Lab Chip. 2004; 4(2):131-5; Petersson et al., “Continuous separation of lipid particles from erythrocytes by means of laminar flow and acoustic standing wave forces” Lab Chip. 2005; 5(1):20-2; and Cushing et al., “Reducing WBC background in cancer cell separation products by negative acoustic contrast particle immuno-acoustophoresis” Anal Chim Acta. 2018; 1000:256-64.

In one embodiment, the present invention contemplates a cell-NACP complex comprising an acoustic contrast factor determinant that is greater than zero and an unbound NACP comprising an acoustic contrast factor determinant that is less than zero.

Specific advantages of the presently disclosed acoustic enrichment technology takes advantage of several recent advances that provide a unique, highly efficient, and versatile approach to remove unbound NACPs. Although it is not necessary to understand the mechanism of an invention, it is believed that an acoustofluidic device significantly shortens the time necessary to prepare adoptive cell compositions as compared to methods to prepare CAR T-cells. In one embodiment, the presently disclosed NACPs are designed to bind mononuclear cells and undergo rapid purification in an acoustic standing wave. This technology is analogous to magnetic-activated cell sorting (MACS), but in a very simple, continuous flow system that obviates the needs for magnetic labels and can be modified to support high throughput processing. Another advantage of this technology is that bound and unbound NACPs experience forces in opposite directions, unlike magnetic separation, which allows for higher purity separations.

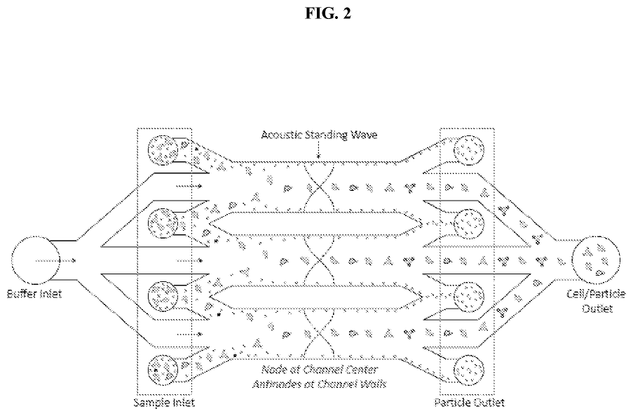

In one embodiment, the present invention contemplates a multi-node acoustofluidic device configured to support high throughput sample processing. See, FIG. 2. In one embodiment, the acoustofluidic device comprises a single transducer. In one embodiment, the single transducer generates a half-wavelength acoustic standing wave in each parallel channel. In one embodiment, samples are produced with >95% separation purities at a rate of up to 1×10⁶ cells/minute across all channels in the device.

In one embodiment, the present invention contemplates an acoustofluidic instrument to purify mononuclear cells for adoptive cellular transfer. It has been shown that acoustofluidic devices can be made using lithographic techniques, including photolithography, deep reactive ion etching, and anodic bonding. Shields I V et al., “Fabrication and Operation of Acoustofluidic Devices Supporting Bulk Acoustic Standing Waves for Sheathless Focusing of Particles” J Vis Exp. 2016(109). Additionally, it has been shown that microfluidic devices with a single channel supporting a half-wavelength acoustic standing wave can purify 10,000 cells per second with efficiencies near 95%. Shields I V et al., “Magnetic separation of acoustically focused cancer cells from blood for magnetographic templating and analysis” Lab Chip. 2016; 16(19):3833-44.

In one embodiment, the acoustofluidic instrument is a multi-parallel acoustofluidic device comprising a plurality of channels. In one embodiment, the device comprises three (3) channels to process 0.3×10⁶ cells per minute. In one embodiment, the device comprises ten (10) channels to process 1.0×10⁶ cells per minute. See, FIG. 2; and Shields I V et al., “Translating microfluidics: Cell separation technologies and their barriers to commercialization” Cytometry B Clin Cytom. 2017; 92(2):115-25.

In one embodiment, an acoustofluidic enrichment instrument comprises a single piezoelectric transducer that forms standing waves in ten (10) parallel microchannels. Due to the high acoustic impedance of silicon and borosilicate glass (primary components of the acoustofluidic devices), it is feasible to create standing waves in parallel channels if the transducer is securely affixed to the base of the silicon device. Assuming a microchannel width of 1,200 μm and an interchannel spacing of 400 μm, the width of the multi-parallel acoustofluidic channels should not exceed 12.4 mm, which is sufficiently smaller than the width of most commercial single array piezoelectric transducers.

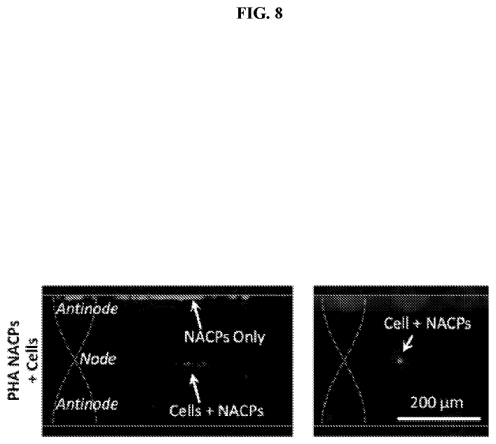

In one embodiment, the acoustofluidic instrument comprises a single channel. The data presented herein shows that mononuclear cells and PHA-based NACPs focus along the pressure node and antinodes, respectively. See, FIG. 4B,C. Fluorescent samples were injected into the device at 100 μL/min. Due to their differential acoustic contrast factors ((φ), particles and cells experience different magnitudes of force, described by the primary acoustic radiation force. Since NACPs experience force in one direction and cells in different direction, the maximum number of NACPs that can bind to a mononuclear cell without forcing cellular acoustophoresis to the pressure antinodes were calculated. Assuming an average volume and density for both mononuclear cells and PHA-based NACPs (50 μm³) six (6) NACPs can bind to one mononuclear cell without pulling it to the pressure antinodes (Vₚ<(V_{c}*φ_{c})/φₚ). The data presented herein shows complete acoustic separation of unbound NACPs; consequently, it is apparent that most mononuclear cells bind less than six (6) NACPs, See, FIG. 8.

Standard monolithic techniques including, but not limited to, photolithography, deep reactive ion etching, anodic bonding have been used to manufacture the silicon-based acoustofluidic devices. Multiple device designs include, but are not limited to: (i) channel widths between 400-1,200 μm; and (ii) channel lengths between 3-20 cm. A piezoelectric transducer is adhered to the bottom of the chip to provide a resonant frequency commensurate with the channel width selected. Resonant frequencies can be determined following the equation c=λf, where c is the speed of sound, λ is the acoustic wavelength, and f is the frequency of the ultrasonic transducer. See, FIG. 4A.

PHA-based NACPs can encapsulate fluorophores and bind to primary mononuclear cells by known methods. Samples can be added to the device where a trifurcating channel collects mononuclear cell:NACP complexes from the center channel outlet and unbound (free) NACPs from the side channel outlets. The data presented herein shows that unbound NACP is indeed separate from cell-bound NACPs. See, FIG. 8. Flow cytometry can validate both the purity and efficiency of particle sorting.

In one embodiment, the present invention contemplates a method comprising administering a pharmaceutically acceptable composition of acoustically purified mononuclear cell:NACP complexes that provide a superior therapeutic outcome as compared to a composition comprising mononuclear cell:NACP complexes and free NACPs.

### IL. Pediatric Lymphomas

Non-Hodgkin's lymphoma (NHL) is the fifth most diagnosed cancer in children under the age of 15, and its incidence is escalating. Reiter A., “Non-Hodgkin lymphoma in children and adolescents” Klin Padiatr. 2013; 225 Suppl 1:S87-93. Diffuse large B-cell lymphoma (DLBCL) is the most common subtype of NHL. While 5-year survival rates for DLBCL range from 60% to 70% after first-line treatment, up to 50% of patients become refractory to, or relapse after, treatment. Crump et al., “Outcomes in refractory diffuse large B-cell lymphoma: results from the international SCHOLAR-1 study” Blood 2017; 130(16):1800-8.

CD19-targeted CAR T-cells are a common treatment for patients with refractory DLBCL. Schuster et al., “Chimeric Antigen Receptor T Cells in Refractory B-Cell Lymphomas” N Engl J Med. 2017; 377(26):2545-54; Neelapu et al., “Axicabtagene Ciloleucel CAR T-Cell Therapy in Refractory Large B-Cell Lymphoma” N Engl J Med. 2017; 377(26):2531-44; Kochenderfer et al., “Long-Duration Complete Remissions of Diffuse Large B Cell Lymphoma after Anti-CD19 Chimeric Antigen Receptor T Cell Therapy” Mol Ther. 2017; 25(10):2245-53; and Turtle et al., “Immunotherapy of non-Hodgkin's lymphoma with a defined ratio of CD8⁺ and CD4⁺ CD19-specific chimeric antigen receptor-modified T cells” Science Translational Medicine. 2016; 8(355):355ra116.

A recent retrospective study, however, revealed that the objective response rate for these patients was only 26% and the median overall survival (OS) was 6.3 months. Relapse after CAR T-cell therapy is often associated with loss of the target antigen. Xu et al., “Mechanisms of Relapse After CD19 CAR T-Cell Therapy for Acute Lymphoblastic Leukemia and Its Prevention and Treatment Strategies” Front Immunol. 2019; 10:2664. Further, CAR T-cell therapy in some tumors has been associated with a T-cell-suppressive microenvironment due to the presence of TAMs and other myeloid-derived suppressor cells. Betsch et al., “Myeloid-derived suppressor cells in lymphoma: The good, the bad and the ugly” Blood Rev. 2018; 32(6):490-8.

CAR T-cell therapy for DLBCLs suffer from three key challenges:
1. Slow and expensive: Isolating, genetically modifying, and expanding T cells usually requires 4-8 weeks. Schultz et al., “Driving CAR T cell translation forward” Science Translational Medicine. 2019; 11(481):eaaw2127. This timeframe is problematic for children with tumors that are not chemo-responsive. Additionally, CAR T-cell therapy usually costs over $500,000 per patient, including the cost of supplies and staff, which is cost-prohibitive for many patients or institutions. Hay et al., “CAR T-cells: costs, comparisons, and commentary” J Med Econ. 2019; 22(7):613-5. For comparison, chemotherapies usually cost about one tenth as much (e.g., $30 k-50 k). Kruse et al., “Administration costs of intravenous chemotherapy in treating peripheral T-cell lymphoma” J Med Econ. 2014; 17(7):446-58.
2. Poor efficacy in solid tumors due to a suppressive microenvironment: Tumor-infiltrating immune cells can be pressured by tumors cells to suppress T cell function, rendering the CAR T-cells ineffective. Long et al., “Reduction of MDSCs with All-trans Retinoic Acid Improves CAR Therapy Efficacy for Sarcomas” Cancer Immunol Res. 2016; 4(10):869-80. These myeloid derived suppressor cells contribute to immune checkpoint expression on both antigen-presenting cells and T cells, suppressive cytokine exposure, and T cell exhaustion. D'Aloia et al., “CAR-T cells: the long and winding road to solid tumors” Cell Death Dis. 2018; 9(3):282.
3. Reliant on the knowledge and presence of tumor-associated antigens: For reasons stated above, CAR T-cell therapy and other antigen-specific immunotherapies require both the knowledge and expression of tumor-associated antigens. In scenarios where antigens are lacking or there is loss of targetable tumor antigen (e.g., CD19-negative relapse), these therapies are ineffective. Sotillo et al., “Convergence of Acquired Mutations and Alternative Splicing of CD19 Enables Resistance to CART-19 Immunotherapy” Cancer Discov. 2015; 5(12):1282-95.

Other treatments for DLBCL for non-responsive lymphomas include high dose “salvage” chemotherapies and stem cell transplantation. While effective in some cases, these strategies carry a risk of long-term morbidity and mortality. One potential strategy for lymphoma is the use of systemic cytokine therapy (CT) to alter the immune microenvironment from protumoral to antitumoral. The advantage of CT is the ease of application and induction of appropriate immune effector cells to spur tumor rejection. However, this approach leads to excessive off-target effects (e.g., pneumonitis, colitis, and immune attacks on healthy tissues, imposing strict dosing constraints that limit therapeutic effects. Gisselbrecht et al., “Improving Second-Line Therapy in Aggressive Non-Hodgkin's Lymphoma” Semin Oncol. 2004; 31 Suppl 2:12-6; Kaplan et al., “Phase II study of recombinant human interferon gamma for treatment of cutaneous T-cell lymphoma” J Natl Cancer Inst. 1990; 82(3):208-12; Shimabukuro-Vornhagen et al., “Cytokine release syndrome” J Immunother Cancer. 2018; 6(1):56; Baldo B A., “Side effects of cytokines approved for therapy” Drug Saf. 2014; 37(11):921-43.

### III. Tumor-Associated Macrophage (TAM)-Mediated Therapy

Macrophages are believed to play a role in many biological processes including, but not limited to, defending against foreign pathogens, wound healing, and/or regulating tissue homeostasis. Driving this versatility is their phenotypic plasticity, which enables macrophages to respond to subtle cues in tractable and tightly measured ways. Macrophages rely on cues from the surrounding tissue microenvironment to guide their polarization into an appropriate phenotype. However, failure of this process can exacerbate many diseases, especially in solid tumors like lymphoma.

For example, the contribution of tumor-associated macrophages (TAMs) to cancer progression is dependent upon their polarization. Macrophage polarization is best characterized by a multidimensional spectrum but can be simplified into an M1/M2 dichotomy. Martinez et al., “The M1 and M2 paradigm of macrophage activation: time for reassessment” F1000prime reports. 2014; 6. M1-like macrophages are classically activated and exhibit proinflammatory effects, defined by the secretion of inflammatory cytokines and reactive oxygen species (ROS), thereby preventing tumor growth. Conversely, M2-like macrophages are alternatively activated and exhibit anti-inflammatory effects, defined by high expressions of scavenging molecules, mannose receptors, and polyamines. Macrophages with M2-like phenotypes in neoplastic tissues have been linked to tumor growth and metastasis. Consequently, repolarizing TAMs toward M1-like phenotypes has gained substantial attention in recent years due to their potential to reject tumors with low immunogenicity in an antigen-agnostic fashion. Because TAMs can account for more than 25% of lymphatic tumors by mass in a patient (e.g., children and/or young adults), this prevalence highlights their potential to treat refractory or relapsed lymphoma. Lee et al., “Targeting of M2-like tumor-associated macrophages with a melittin-based pro-apoptotic peptide” 2019; 7(1):147; Poh et al., “Targeting Macrophages in Cancer: From Bench to Bedside’ Front Oncol. 2018; 8:49; and Steidl et al. “Tumor-associated macrophages and survival in classic Hodgkin's lymphoma” N Engl J Med. 2010; 362(10):875-85.

In one embodiment, the present invention contemplates a system for rapid adoptive cell transfer for patients with antigen-limited lymphomas. For example, healthy mononuclear cells are isolated from donors and then attached to negative acoustic contrast particles (NACPs). Subsequent to purifying mononuclear cell-NACP complexes from unbound NACPs using a high-throughput acoustofluidic device the purified mononuclear cell-NACP complexes are administered to a lymphoma patient intravenously (IV).

Mononuclear cells are members of an innate immune system, consequently they can migrate to areas in the body that are otherwise difficult to access. Anselmo et al., “Monocyte-mediated delivery of polymeric backpacks to inflamed tissues: a generalized strategy to deliver drugs to treat inflammation” J Control Release. 2015; 199:29-36.

However, a major disadvantage to use mononuclear cells as part of a particle drug delivery platform is the concomitant co-administration of particles without bound drugs. These uncomplexed particles primarily accumulate in the liver and spleen, generating adverse effects. These side effects can drastically reduce the maximum tolerated dosage (MTD) of drug, thus suppressing or eliminating therapeutic benefits. Current approaches to remove unbound particles include differential centrifugation and magnetic separation. Differential centrifugation is slow, tedious, and offers poor separation efficiencies (e.g., 50-90%). On the other hand, magnetic separation is more rapid and efficient, typically generating >95% separation efficiencies of unbound particles. Shields I V et al., “Microfluidic cell sorting: a review of the advances in the separation of cells from debulking to rare cell isolation” Lab Chip. 2015; 15(5):1230-49. Magnetic separation, however, requires the use of magnetic particles on cells, which can be toxic at high concentrations, hampering their clinical and translational value. Shields I V et al., “Materials for Immunotherapy” Adv Mater. 2019:e1901633.

Mononuclear cell-NACP therapy has specific advantages over CAR T-cell therapy including, but not limited to:
1. Rapid and Inexpensive: Mononuclear cells are a subset of the innate immune system with the ability to migrate to areas of inflammation. By attaching NACPs to mononuclear cell surfaces to delivery immunomodulatory payloads, the time to treatment is accelerated from several weeks to several hours, because it bypasses the need for: (i) autologous cells; (ii) genetic modification; and (iii) T cell expansion. Instead, this system is designed to use stored mononuclear cells from healthy donors, attached them to NACPs in a single batch, and then purify NACP-mononuclear cell complexes using a rapid and cost-effective acoustofluidic device.
2. Reprograms Immunosuppressive Tumor Microenvironments: The therapeutic mechanism of the proposed system includes, but is not limited to:
   i) delivering a CCL1/CCL2 modulatory drug (e.g., TMP195; a class IIA histone deacetylase (HDAC) inhibitor that induces proinflammatory phenotypes by modifying CCL1 and CCL2 expression and induces a durable phenotypic shift of TAMs toward antitumor (M1-like) phenotypes. Several M1-macrophage polarizing agents have potentiated similar phenotypic shifts to promote tumor rejection, establishing precedence for this approach; Guerriero et al., “Class IIa HDAC inhibition reduces breast tumours and metastases through anti-tumour macrophages Nature. 2017; 543(7645):428-32; Cassetta et al., “Targeting macrophages: therapeutic approaches in cancer” Nature Reviews Drug Discovery. 2018:1-18; and Yue et al., “Biomimetic Nanoparticles Carrying a Repolarization Agent of Tumor-Associated Macrophages for Remodeling of the Inflammatory Microenvironment Following Photothermal Therapy” ACS Nano. 2021; and
   ii) coating NACPs with CpG ODNs to activate tumor-infiltrating dendritic cells (TIDCs). These mechanism work in concert wherein the CCL1/CCL2 modulatory drug polarizes TAMs toward antitumor phenotypes, clearing tumor tissue and creating neoantigens, and the CpG ODNs recruit and activate TIDCs such that the activated TIDCs educate CTLs. Thus, the presently disclosed therapy leverages both innate and adaptive immune responses to attack solid tumors without a priori knowledge of tumor-associated antigens.
3. Agnostic To Antigen Expression: Success of CAR T-cell therapies depends on the knowledge and presence of tumor-associated antigens. Alternatively, M1-polarized macrophages kill tumor cells where tumor-associated antigens are spare or unknown, giving them a pathway to succeed where CAR T-cell therapies have failed. Lee et al., “Macrophage-based cell therapies: The long and winding road” J Control Release. 2016; 240:527-40.

In one embodiment, the present invention contemplates a method for reprogramming lymphatic tumors thereby immunologically rejecting solid tumors in an antigen-agnostic fashion. For example, clinical efforts have been made to repolarize TAMs via adoptive macrophage transfer, which involved polarizing macrophages ex vivo with IFNγ and injecting autologously into patients. However, these studies failed, as macrophages and TAMs quickly acquired protumoral phenotypes due to the strongly immunosuppressive microenvironment of solid tumors. Tso et al., “Phenotypic and functional changes in blood monocytes following adherence to endothelium” PLoS One. 2012; 7(5):e37091.

This limitation can be overcome by slowly releasing a potent M1-macrophage polarizing drug (e.g., TMP195) from NACPs bound to mononuclear cells following deep infiltration into lymphatic tumors. Accumulated NACPs serve as a depot to potentiate a more durable shift in TAM repolarization as compared to previous studies. It has been shown that cells coated with M1-macrophage polarizing particles not only maintained their phenotypes better than cells without M1-macrophage polarizing particles, but that these particle-cell complexes potentiated a prolonged shift in TAMs toward M1-like phenotypes and slowed tumor growth. Shields et al., “Cellular backpacks for macrophage immunotherapy” Science Advances. 2020; 6(18):eaaz6579.

In one embodiment, the present invention contemplates a cancer vaccine comprising NACPs coated with a TLR9 agonist (e.g., CpG ODNs) and an M1-macrophage polarizing compound. TLR9 agonists have been reported to activate tumor infiltrating dendrictis cells (TIDCs). Macrophages display vastly enhanced phagocytic behaviors when stimulated by M1-macrophage polarizing compounds by forming neoantigens in the tumor microenvironment, while activated TIDCs display increased MHC expression thereby improving antigen uptake and presentation. Lewis et al., “Distinct role of macrophages in different tumor microenvironments” Cancer Res. 2006; 66(2):605-12. Neoantigen-displaying DCs then migrate to the draining lymph nodes to educate naïve T cells and form cytotoxic T lymphocytes (CTLs). An immunization method comprising the cancer vaccine further comprises intraperitoneal (IP) injections of an anti-PD-1 checkpoint inhibitor to improve the infiltration of CTLs into lymphatic tumors. See, FIG. 1C. In one embodiment, the cancer vaccine improves tumor clearance. In one embodiment, the cancer vaccine induces protective immunity against molecularly similar tumors.

### EXPERIMENTAL

### Example I

### Preparation of Negative Acoustic Contrast Particles

NACPs can be prepared with different sizes, shapes, and surface coatings to maximize binding to primary mononuclear cells and minimize phagocytosis.

NACPs will encapsulate TMP195, a potent M1-polarizer of TAMs and display CpG oligodeoxynucleotides (ODNs; TLR9 agonists) on their surfaces.

Optimal drug loading of the NACPs is determined with TAM cultures in vitro, and phenotypic expression of M1-associated macrophage markers (CD80/86, iNOS).

### Example 11

### In Vivo NACP Administration

NACP-cell samples can be infused into mice bearing CD19-sparse lymphoma. Sample biodistributions will be quantified. Efficacy of drug delivery and NACP-cell complex infiltration is determined by TAM polarization, DC activation, and cytotoxic T lymphocyte (CTL) infiltration.

### Example III

### Acoustofluidic Devices

Acoustofluidic device designs (dimensions, frequency) are used that achieve a 95% removal of unbound NACPs.

Operational parameters (e.g., sample concentration, pressure amplitude, flow rate) will be varied to achieve high purity separations. Parallel microchannels will be tested to evaluate feasibility of scale-up to clinical volumes.

### Example IV

### In Vivo Administration of Acoustically Enriched NACPs

Acoustically purified samples will be infused into mice with CD19-sparse lymphoma at different concentrations. Biodistribution and tumor-associated immune cells will be phenotyped and compared to non-acoustically purified controls. Survival studies will be performed with escalating drug dosages to determine overall efficacy with and without the aid of anti-PD-1.

### Example V

### NACP-CpG ODN Coatings

This example demonstrates coating of an NACP surface with CpG ODN to activate primary dendritic cells isolated from the spleen using a negative cell selection kit (Miltenyi).

One subtype of CpG that promotes DC maturation for lymph node migration is CpG 1585 which has an MTD of 20 mg/kg. Yamamoto et al., “Class A CpG Oligonucleotide Priming Rescues Mice from Septic Shock via Activation of Platelet-Activating Factor Acetylhydrolase” Front Immunol. 2017; 8:1049. Assuming 0.1-20% of the injected NACPs arrive at the tumor site, coatings are created that corresponding to 0.04-6.0 wt. % CpG. These CpG-coated NACPs are co-cultured with DCs and the cells are phenotyped after 24, 48, and 72 hrs.

Flow cytometry is used to assess molecular expression of MHCII and costimulatory molecules, such as CD80/86. Release of T cell activating cytokines are also measured with ELISA (e.g., IL-12, TNFα). DC viability can be monitored using live/dead cell stains for quantification by flow cytometry.

### Example VI

### NACP Biodistribution

This example assesses how an increasing number of NACPs bound per cell affects biodistribution. The data presented herein shows that monocytes can infiltrate inflamed tissues with particle-modified surfaces. See, FIG. 5. Consequently, it is expected that mononuclear cell-mediated infiltration becomes reduced with an increasing number of NACPs bound to their surfaces.

Mononuclear cells with increasing numbers of surface bound NACPs are created by incubating ˜1×10⁶ mononuclear cells with increasing ratios of NACPs (i.e., 1:1, 1:3, 1:9). The average number of NACPs bound per mononuclear cell can then be quantitated after 2 hrs of incubation.

Mononuclear cells are discriminated from endogenous cells with the VivoTrack 680 stain and NACPs are labeled with Nile red. Once prepared, the differentially labeled mononuclear cell cell-NACP mixtures with, or without, acoustofluidic purification, are infused through the lateral tail vein of mice burdened with CD19-negative A20 tumor.

CD19-negative A20 tumors are established by culturing CD19-negative A20 cells and injecting 5×10⁶ cells subcutaneously into immunocompetent BALB/c mice, representing tumor antigen-poor tumors. Otero et al., “Cd19-dependent activation of Akt kinase in B-lymphocytes” J Biol Chem. 2001; 276(2):1474-8; Donnou et al., “Immune adaptive microenvironment profiles in intracerebral and intrasplenic lymphomas share common characteristics” Clin Exp Immunol. 2011; 165(3):329-37; and Palmieri et al., “In vivo targeting and growth inhibition of the A20 murine B-cell lymphoma by an idiotype-specific peptide binder. Blood. 2010; 116(2):226-38. doi: 10.1182/blood-2009-11-253617.

Approximately 48 hrs after infusion, mice are euthanized and the primary organs (i.e., liver, lungs, spleen, heart, brain, kidney, tumors) extracted, weighed, homogenized, and fluorescently analyzed by a plate reader to quantify VivoTrack 680 and Nile red. This method quantifies the relative targeting efficiency of mononuclear cells without and with bound NACPs as a function of NACPs per mononuclear cell.

### Example VII

### NACP Activation of Immune Cells

This example demonstrates NACP-mediated TAM repolarization. Dosing studies are performed to identify NACP concentrations per injection to facilitate a durable repolarization of TAMs with minimal toxicity. As the number of NACPs per mononuclear cell are fixed, the dose is controlled by the total number of mononuclear cells injected per mouse. Mice expressing CD19-depleted A20 cell tumors are used in accordance with Example VI.

Fourteen (14) days after inoculation, each mouse receives one injection of mononuclear cell-NACPs with, or without, acoustic purification. Escalating doses are given, corresponding to 1, 5, 15, and 50 mg/kg TMP195 and 0.4, 2, 6, and 20 mg/kg CpG (where the highest dose corresponds to the MTDs).

Two days after injection, 50 μL of peripheral blood is collected from the submandibular venous plexus and screened for serum levels of TNFα, IL-1β, IL-6, IL-12, and IL-18 to assess toxicity.

Seven days after injection, mice are sacrificed, and tumors will be harvested following established methods. Tumors are digested using collagenase, DNase and phenotypically profiled by flow cytometry. TAMs and mononuclear cells are isolated using a negative selection kit and screened for molecular expression to determine polarization (i.e., CD80/86, iNOS, Arg-1, and VEGF).

DCs will also be isolated and screened for the activation marker MHCII and costimulatory molecules CD80/86. The relative expression of each marker will be determined by comparing to control mice treated with saline.

Tumor sections will be taken from mice in each cohort for histopathological staining to identify TAMs by CD68⁺ and DCs by CD11 b⁺/CD11c⁺ to resolve the spatial distribution of tumor-resident and tumor-infiltrating immune cells. Al Faraj et al., “Real-time high resolution magnetic resonance tracking of macrophage subpopulations in a murine inflammation model: a pilot study with a commercially available cryogenic probe” Contrast Media Mol Imaging. 2013; 8(2):193-203.

It is expected that NACPs will stably bind to mononuclear cells and that >50% of bound NACPs will evade phagocytosis for >48 hrs. It is also expected that the NACPs with encapsulated TMP195 and coatings of CpG: (i) promote polarization of BMDMs toward M1-like phenotypes, even in tumor-mimicking conditions; and (ii) activate DCs to upregulate MHC and costimulatory molecules. Mononuclear cells with just one NACP bound to its surface (on average) are expected to infiltrate tumors with the greatest efficiency. It is also expected that the highest dose of TMP195 and CpG to elicit the most pronounced response in TAM repolarization and DC activation.

### Example VIII

### Acoustofluidic Purification of Mononuclear Cell:NACP Complexes

This example describes that separation of unbound NACPs that are not attached to a mononuclear cell from bound NACPs are bound to at least one mononuclear cell to create a highly purified mononuclear cell-NACP composition. Previous studies have shown that IV-injected particles that are not attached to a targeting moiety generally accumulate in the liver and spleen, leading to adverse effects that constrain dosing and limit overall therapeutic efficacy. A distinguishing feature of the presently disclosed acoustofluidic enrichment system is its ability to rapidly separate unbound particles from cell-bound particles.

An acoustofluidic instrument as described herein capable of processing up to 1×10⁶ cells/minute, which is of high clinical relevancy, as patients with DLBCL usually receive 0.4-8.2×10⁶ CAR T-cells/kg. Park et al., “CD19-targeted CAR T-cell therapeutics for hematologic malignancies: interpreting clinical outcomes to date” Blood. 2016; 127(26):3312-20. If a patient is 45 kg (average weight of a 12-year-old child), they would require ˜200×10⁶ CAR T-cells. With the proposed acoustofluidic enrichment instrument, unbound particles can be separated from the required number of NACP:mononuclear cell complexes in under 4 hours. This timeline is >100× faster than that of CAR T-cell therapy, which takes between 4-8 weeks. It is expected that the presently disclosed acoustofluidic enrichment instrument safely removes >95% of unbound NACPs from mononuclear cell-NACP complexes at rates up to 1×10⁶ cells/minute.

### Example IX

### Acoustofluidic Enrichment Device Operation

This example describes acoustofluidic device operating parameters that generate high purity mononuclear cell:NACP complex compositions.

PHA-based NACPs are fluorescently labeled and incubated with primary mononuclear cells for 2.0 hrs to permit binding. Sample mixtures are then be added to the acoustofluidic enrichment device which is operated within the following parameters: (i) flow rate between 25-250 μL/min; (ii) sample concentration between 0.4×10⁶−4.0×10⁶ cells/mL); and (iii) acoustic pressure amplitude between 10 kPa-1.0 MPa. Barnkob et al., “Measuring the local pressure amplitude in microchannel acoustophoresis” Lab Chip. 2010; 10(5):563-70.

Flow rates and sample concentrations are adjusted to produce a constant throughput of 0.1×10⁶ cells/min. Although it is not necessary to understand the mechanism of an invention, it is believed that acoustofluidic separation performance diminishes with high sample concentrations due to scattering, and even though higher flow rates maintain elevated throughput it also diminishes separation purity.

It is also believed that pressure linearly increases in proportion with acoustic force; however, pressures that are too high can generate resistive heating which impairs separation and affects cellular viability. Muller et al., “A numerical study of microparticle acoustophoresis driven by acoustic radiation forces and streaming-induced drag forces” Lab Chip. 2012; 12(22):4617-27. In one embodiment, an acoustofluidic enrichment instrument produces a high purity mononuclear cell-NACP complex at <40° C.). Further, the high acoustic pressure amplitudes for long durations may generate significant heating in the microchip, which can be deleterious to cell viability. If this occurs, a Peltier temperature control system can be integrated to the device using a high thermal conductivity aluminum stage. Augustsson et al., “Automated and temperature-controlled micro-PIV measurements enabling long-term-stable microchannel acoustophoresis characterization” Lab Chip. 2011; 11(24):4152-64.

### Example X

### Survival Studies

Mice will be randomly assigned into groups receiving 1, 3, or 5 cell transfers, separated by 5 days. Half of the mice in each cohort will also receive IP injections of anti-PD-1 two days after each cell transfer.

Survival groups include: (i) saline, (ii) mononuclear cells, (iii) mononuclear cell:NACP complexes without drug, (iv) mononuclear cell:NACP complexes with TMP195, (v) mononuclear cell:NACP complexes with CpG and TMP195, and (vi) group “v” with anti-PD-1.

Mice will be monitored every day until either 90 days post-inoculation or terminal endpoints are reached (e.g., >15% weight loss, ulcer formations, or immobility), whichever occurs first.

### Example XI

### Mechanism of Action Study

This example assesses a possible therapeutic mechanism of action by profiling tumor-associated immune cells.

Mice are randomized into the same groups in accordance with Example X. Five days after the last infusion (i.e., 10, 20, or 30 days after the first therapeutic injection for mice receiving 1, 3, or 5 cell infusions), mice will be euthanized, and their tumors will be harvested. Tumors are digested using collagenase, DNase and EpCAM⁺ cells. EpCAM is expressed on A20 cells, using a negative cell selection isolation kit. Ziegler et al., “EpCAM, a human tumor-associated antigen promotes Th2 development and tumor immune evasion” Blood. 2009; 113(15):3494-502.

Tumor-associated CD45⁺ leukocytes are screened for: i) CD11b/CD11c DC activation markers including, but not limited to, CD28, CD80/86, MHCII); ii) T-cell markers including, but not limited to, CD3, CTL, CD8, CD25, CD69; and iii) CD 68 TAMs and polarization markers including, but not limited to, CD80/86, iNOS, Arg-1, VEGF). Rabb H., “The T cell as a bridge between innate and adaptive immune systems: implications for the kidney” Kidney Int. 2002; 61(6):1935-46. The lungs are also isolated which undergo histopathological staining to identify metastases.

Since the acoustically purified cell samples will have fewer unbound NACPs, it is expected that a greater proportion of drug reach the tumors than an unpurified composition, providing a means to increase drug activity and reduce off-target toxicity. It is also expected that a single channel and a ten channel acoustofluidic device removes >95% of unbound NACPs while maintaining throughputs of 0.1×10⁶ cells/minute. It is expected that acoustically purified cell samples result in a higher percentage of NACPs infiltrating solid tumors (i.e., >7% of total NACPs infiltrating tumors) and that this value will be significantly higher than non-purified cell samples. It is also expected that acoustically purified cell samples repolarize TAMs more strongly than controls of non-acoustically purified cell samples and/or saline-treated mice, as indicated by a significant increase in the biomarkers including, but not limited to, iNOS, CD80/86, and MHCII and a significant decrease in CD206. It is also expected that anti-PD-1 administration enhances CTL infiltration into tumors.

Finally, an optimal dose and treatment schedule results in significantly improved animal survivals, as indicated by a log-rank test, and tumor eradication producing immunological memory against tumor cell rechallenges.

### Example XII

### PDMS Template Preparation

PDMS templates will be prepared using soft lithography by methods similar to those described previously. Zhang et al., “Fabrication of multilayered microparticles by integrating layer-by-layer assembly and microcontact printing’ Small. 2011; 7(21):2998-3004.

Briefly, silicon molds will be fabricated by monolithic photolithography and passivated with a thin film of trichloro(1H,1H,2H,2H-perfluorooctyl)silane (FDTS) by vapor deposition. A 10:1 weight ratio of PDMS base to crosslinker from a Sylgard 184 kit will be mixed and poured on top of the silicon molds in separate Petri dishes (˜20 g per mold). PDMS will be degassed in a desiccator at 25° C. until no visible bubbles remain. Dishes will be placed in an oven at 65° C. overnight to cure the PDMS. After curing. PDMS templates will be removed from the molds by cutting the Petri dishes and peeling away the PDMS. See, FIG. 3B.

### Example XIII

### Cell-Adhesive LBL Coatings

Cell-adhesive coatings will be made using known procedures described below. Briefly, a 2 mg/mL aqueous solution of hyaluronic acid (HA) will be prepared in 150 mM NaCl (pH=6.8), and a 2 mg/mL aqueous solution of poly(allylamine) hydrochloride (PAH; 17.5 kDa) will be prepared in 150 mM NaCl (pH=6.8). The HA, PAH, and 150 mM NaCl (pH=6.8) solutions will be separately poured into weigh boats.

PDMS templates will be rinsed with isopropyl alcohol and dried by a steady stream of air. Templates will then be placed patterned side down in the HA solution for 15 min. Care will be taken to ensure the templates float to maximize contact of the PDMS with the solution. Templates will be transferred to the NaCl solution for 2 min and then rinsed with DI water to remove free HA. Templates will be transferred to the PAH solution for 15 min in the same fashion, then transferred to new weigh boats with the NaCl solution for 2 min. Templates will be rinsed with DI water, and the entire process will be repeated once more to form a layer-by-layer (LBL) coating of HA/PAH/HA/PAH. Coated templates will be rinsed with DI water and dried by a stream of air.

### Example IXV

### Microdisc Fabrication

An 8% w/v solution of PHA in acetone will be prepared from a 100:1 weight ratio of nonfluorescent PHA (7-17 kDa) and fluorescent PLGA (10-30 kDa, LG 50:50 rhodamine B; PolySciTech) to enable facile visualization. TMP195 will be dissolved directly into solution at the appropriate concentrations as described above. PDMS templates will be cut into quadrants and spin-coated with 225 μL PHA solution per quadrant at 2,000 rpm for 20 sec (at a 200 rpm/sec ramp). In parallel with this step, poly(vinyl alcohol) (PVA)-coated dishes will be prepared by making a 3% w/v solution of PVA (13-23 kDa, 87% hydrolyzed) in DI water. The solution will be stirred at 80° C. for several hours, and excess crystals will be filtered using a 0.22 μm filter. Sterile Petri dishes will be coated with 2.5 mL of solution, placed in an oven at 60-75° C. until dry.

Microdiscs will then be printed using techniques like those described previously. Xia et a., “Asymmetric biodegradable microdevices for cell-borne drug delivery” ACS Appl Mater Interfaces. 2015; 7(11):6293-9. Briefly, a beaker will be filled with DI H2O and heated to 65° C. The coated side of a PVA-coated dish will be held ˜2 cm over the beaker for 6-12 sec. A PDMS quadrant containing the PHA/TMP195 solution will be immediately pressed onto the warmed PVA dish and consistent pressure will be applied for 15-20 sec. The quadrant will then be peeled away, leaving a coating of discoidal particles on the dish. See, FIGS. 3A-3D. This will be repeated until the material fully transfers. Particles will be stored at −80° C. until needed. To harvest the particles, dishes will be wetted with 2.5 mL of PBS and gently washed. This will be repeated until the surface appeared mostly clear (typically twice). The solution will be collected, passed through a 40 μm cell strainer, and centrifuged at 2,500×G for 5 min. PHA particles will be resuspended in 5 ml of bone marrow media (BMM⁻; i.e., 500 ml of DMEM F12, 50 ml of FBS, 5 ml of Pen Strep, and 25 ml of 200 mM GlutaMAX) or serum-free BMM⁻(i.e., BMM⁻ sans FBS), depending on the application.

### Example XV

### Bone Marrow Isolation

Progenitor cells will be isolated from murine bone marrow following previous methods. Zhang et al., “The isolation and characterization of murine macrophages” Curr Protoc Immunol. 2008; Chapter 14:Unit 14 1.

Briefly, 6-8-week-old BALB/c mice will be euthanized by CO₂ inhalation. Sterile surgical scissors will be used to extract the tibias, femurs, and humeri. Isolated bones will be submerged in 70% ethanol, rinsed with PBS, and then transferred to a separate PBS solution. In a sterile environment, epiphyses of each bone will be cut, and the bones will be flushed with PBS via a syringe with a 31 G needle into a 50 mL collection tube. solution will be mixed thoroughly, passed through a 40 μm cell strainer, and centrifuged at 350×G for 10 min at 4° C. Cells will be resuspended in Bambanker (2 mL/mouse equivalent; Lymphotec, Inc.) and stored in cryovials at −80° C. until needed. Methods to isolate of other populations of primary cells from BALB/c mice (i.e., monocytes, DCs, T cells) will be followed from the manufacturer of those cell isolation kits (e.g., Miltenyi).

### Example XVI

### Bone Marrow-Derived Macrophage (BMDM) Culture

BMDMs will be differentiated and cultured from murine bone marrow progenitor cells following methods described previously. Evans et al., “Macrophage mediated delivery of light activated nitric oxide prodrugs with spatial, temporal and concentration control” Chem Sci. 2018; 9(15):3729-41.

Briefly, frozen bone marrow will be thawed and mixed with 4° C. BMM⁻ at 1:5 ratio by volume. The solution will be centrifuged, the liquid will be aspirated, cells will be resuspended in BMM⁺ (i.e., BMM⁻ with 20 ng/mL macrophage-colony stimulating factor, M-CSF), and cells will be counted with a hemocytometer. Approximately 4×106 bone marrow cells will be added to non-tissue culture (TC)-treated T175 flasks containing 25 mL BMM⁺. Cells will be incubated under standard culture conditions. 25 mL additional BMM⁺ will be added to the flasks on Days 3 and 7. On Day 8, media will be aspirated from the flasks, and cells will be washed once with 10 mL PBS. To dislodge the cells, PBS will be aspirated and replaced with 10 mL Accumax (Innovative Cell Technologies) at 4° C. Cells will be incubated with the Accumax at 37° C. for 10 min. The flask will then be removed from the incubator and vigorously thumped several times. More Accumax (10 mL) will be added to the flask, and cells will be incubated for an additional 10 min and thumped again. The suspension of BMDMs will be added to a 50 mL conical tube with an equal volume of BMM- and centrifuged. The supernatant will be aspirated and replaced with BMM⁺. BMDMs will be counted and plated on non-TC-treated well plates (amounts adjusted for 12-well plates) at a concentration of 2.5×10⁵ cells/well in a volume of 1 mL/well and incubated uider standard conditions for 24 h. All centrifugation steps will be performed at 350×G for 10 min at 4° C.

### Example XVI

### Binding Particles to Mononuclear Cells

Particles (either microcontact printed discs or homogenized spheres) will be harvested and centrifuged at 2,500×G for 5 min at 4° C. and resuspended in serum-free BMM⁻. Meanwhile, mononuclear cells will be removed from the incubator, pelleted, and resuspended in serum-free BMM-media. Particles will be counted using a hemocytometer, and a well-defined ratio of particles will be added to mononuclear cells in a conical tube, as described above. Conical tubes will be centrifuged at 300×G for 7.5 min to allow particles and mononuclear cells to gather along the bottom. The supernatant will be aspirated, leaving a small volume behind (<50 μL) for particles and cells to incubate and bind along the bottom. After 2 hrs, serum-free BMM media will be exchanged with BMM-media by resuspension. Samples that will not undergo acoustic separation will be used immediately. Samples that will undergo acoustic separation will be processed as described above.

### Example XVII

### Phenotyping Macrophage Cultures

Serial dilution will be performed to replace media in each well of the 12-well plates with Hank's Balanced Salt Solution (HBSS). Then 500 μL HBSS will be aspirated from each well and replaced with 2 mL Accumax. Plates will be incubated at 37° C. and 5% CO₂ for 10-15 min. Plates will then be thumped to release BMDMs and the respective groups will be collected into separate 50 mL tubes with an equal volume BMM⁻. Cells will be centrifuged, and pellets will be resuspended in 1 mL of stain buffer, comprising 1% FBS in PBS without Mg²⁺ or Ca²⁺ (pH=7.4-7.6). Cells will be transferred into 1.5 mL Eppendorf tubes, where they will be centrifuged again. Pellets will be resuspended in 99 μL of stain buffer with 1 μL Fc block and will be incubated for 15 min at 4° C. After incubation, samples will be diluted with 1 mL stain buffer, centrifuged, and resuspended in 1 mL of stain buffer. Each sample will then split into two groups of 500 μL for surface marker staining and intracellular staining. For surface staining, samples will be centrifuged and resuspended in antibody mixtures as described above at concentrations suggested by the manufacturer in the dark at 4° C. After 30 min, cells will be washed with 1 mL stain buffer, centrifuged, resuspended in 300 μL, and stored in the dark at 4° C. until use. For intracellular staining, samples will be fixed and permeabilized following instructions from the manufacturer (BD Biosciences). Cells will be centrifuged and resuspended in 100 μL of an antibody solution as described above with Penn/Wash™ Buffer at various concentrations in the dark at 25° C. After 30 min, cells will be diluted with 1 mL of Perm/Wash™ Buffer, centrifuged, resuspended in 300 μL of stain buffer, and stored in the dark at 4° C. until use. All centrifugation steps will be performed at 350×G for 10 min at 4° C. Compensation and voltage settings will be determined one day prior using sets of compensation beads, each stained with one antibody. Up to 100,000 events will be collected for each sample. Other immune cells (e.g., monocytes, DCs, T cells) will be isolated and phenotyped following methods as described above

### Example XVIII

### Phenotyping Tumor-Associated Immune Cells

Procedures for isolating and staining tumor-associated immune cells are similar to those described previously. Pusuluri et al., “Role of synergy and immunostimulation in design of chemotherapy combinations: An analysis of doxorubicin and camptothecin” Bioeng Transl Med. 2019; 4(2):e10129.

Briefly, mice will be euthanized via CO₂ inhalation. Primary tumors will be harvested, cut into small pieces (<5 mm thick), and enzymatically degraded using a mouse tumor dissociation kit with a gentleMACS dissociator (Miltenyi Biotec). Cells will be centrifuged and resuspended in ACK red cell lysis buffer supplemented with 50 U/mL of DNAse 1 for 5 min. Cells will again be centrifuged and resuspended in PBS to quantify the remaining intact cells. Leucocytes will be isolated from the general population using a CD45′ isolation kit, following instructions from the manufacturer (Miltenyi Biotec). For the remainder of the phenotyping study, 1×10⁶ cells per tumor will be used and all steps will be performed in 100 μL FACS buffer (PBS with 3% FBS) supplemented with additional reagents as necessary. Cells will be blocked for 30 min in a solution consisting of 5% rat serum, 5% mouse serum, and 1% anti-mouse CD16/32 antibody. Cells will be stained with test and control antibodies for 30 min at 25° C. and for 20 min on ice in a dark enclosed space. Cells will then be washed twice with ice-cold FACS buffer and resuspended in 500 μL PBS. Following instructions from the manufacturer, cells will be stained with a viability stain to measure cell viability at the end of all treatment steps. Stained cells will then be analyzed by flow cytometry. Compensation and voltage settings will be determined one day prior using sets of compensation beads, each stained with one antibody. Up to 100,000 events will be collected for each sample. All centrifugation steps will be performed at 350×G for 10 min at 4° C.

1. A composition comprising a substantially purified population of mononuclear cell:particle complexes that is substantially devoid of unbound particles, wherein said particles comprise an elastomeric polymer and encapsulate an M1-macrophage polarizing drug.

2-3. (canceled)

4. The composition of claim 1, wherein said M1-macrophage polarizing drug is selected from the group consisting of resiquimod, motolimod, GS9620, CEP32496, BLZ945, OS1930, XL228, UNC2025, CELECOXIB, TMP195, TRICHOSTATIN A, IBET151, and INDOXIMOD.

5. The composition of claim 1, wherein said composition further comprises a checkpoint inhibitor.

6-9. (canceled)

10. The composition of claim 1, wherein said particle has an anisotropic shape.

11. The composition of claim 1, wherein said particle is a negative acoustic contrast particle.

12. The composition of claim 1, wherein said mononuclear cell:particle complex has an acoustic contrast factor ((p) of greater than 0.

13. The composition of claim 1, wherein said unbound particles have an acoustic contrast factor (y) that is less than 0.

14. A method, comprising:
a) providing;
i) a plurality of mononuclear cells;
ii) a plurality of elastomeric particles; and
iii) an M1-macrophage polarizing drug;
b) combining said plurality of mononuclear cells, said plurality of elastomeric particles and said M1-macrophage polarizing drug to create a mixture comprising:
i) a plurality of mononuclear cell:elastomeric particle complexes that encapsulate said M1-macrophage polarizing drug, and
ii) a plurality of free, unbound, elastomeric particles that encapsulate said M1-macrophage polarizing drug;
c) passing said mixture through an acoustic enrichment device to create a substantially purified population of mononuclear cell:elastomeric particle complexes that encapsulate said M1-macrophage polarizing drug that is substantially devoid of unbound elastomeric particles comprising said M1-macrophage polarizing drug;
d) administering said substantially purified population of mononuclear cell:elastomeric particle complexes to a patient, such that a portion of said substantially purified population of mononuclear cell:elastomeric particle complexes embeds within a solid tumor, said solid tumor comprising tumor-associated immune cells; and
e) releasing said M1-macrophage polarizing drug from said embedded substantially purified population of mononuclear cell:elastomeric particle complexes, thereby reprogramming said tumor-associated immune cells to reject said solid tumor in an antigen-agnostic manner.

15. The method of claim 14, wherein said solid tumor is a cancer selected from the group consisting of bladder cancer, breast cancer, cervical cancer, colon cancer, endometrial cancer, esophageal cancer, gall bladder cancer, gastric cancer, glioblastoma, head and neck cancer, hepatocellular carcinoma, lymphoma, lung cancer, melanoma, mesothelioma, neuroendocrine cancer, ovarian cancer, pancreatic cancer, prostate cancer, renal cell carcinoma, and sarcoma.

16. The method of claim 14, wherein said mononuclear cells are selected from the group consisting of mononuclear cells, macrophages, neutrophils, dendritic cells, NK cells, B cells, T cells, mast cells, mesenchymal stem cells, and their progenitor cells.

17. The method of claim 14, wherein said M1-macrophage polarizing drug is selected from the group consisting of resiquimod, motolimod, GS9620, CEP32496, BLZ945, OS1930, XL228, UNC2025, CELECOXIB, TMP195, TRICHOSTATIN A, IBET151 and INDOXIMOD.

18. The method of claim 14, wherein said administering further comprises a checkpoint inhibitor.

19-22. (canceled)

23. The method of claim 14, wherein said elastomeric particle has an anisotropic shape.

24. The method of claim 14, wherein said elastomeric particle is a negative acoustic contrast particle.

25. The method of claim 14, wherein said mononuclear cell:particle complex has an acoustic contrast factor (y) of greater than 0.

26. The method of claim 14, wherein said unbound particles have an acoustic contrast factor ((p) that is less than 0.

27. A cancer vaccine composition comprising a plurality of negative acoustic contrast particles coated with a toll-like receptor (TLR) agonist and encapsulating an M1-macrophage polarizing compound.

28. The cancer vaccine composition of claim 27, wherein said TLR agonist is bound to a tumor infiltrating dendritic cell.

29. The cancer vaccine composition of claim 27, further comprising a medium capable of an intravenous (IV) injection or an intraperitoneal (IP) injection.

30. The cancer vaccine composition of claim 27, wherein said cancer vaccine further comprises checkpoint inhibitor.

31-34. (canceled)
